(19) United States
(12) Patent Application Publication
(10) Pub. No.: US 2023/0181448 A1
(43) Pub. Date: Jun. 15, 2023

(54) CATIONIC COSMETIC COMPOSITION FOR REINFORCING ADHESION OF MAKEUP AND PREVENTING RUBBING-OFF THEREOF

(71) Applicant: COSMAX, INC., Gyeonggi-Do (KR)

(72) Inventors: Ji Hyun LEE, Gyeonggi-do (KR); Jun Bae LEE, Gyeonggi-do (KR); Eun Ju SEO, Gyeonggi-do (KR); Myeong Sam PARK, Seoul (KR)

(21) Appl. No.: 17/924,330

(22) PCT Filed: Jun. 15, 2021
(86) PCT No.: PCT/KR2021/007433

§ 371 Date: Nov. 9, 2022

(30) Foreign Application Priority Data
Jun. 25, 2020 (KR) 10-2020-0078047

(51) Int. Cl. A61K 8/73; A61K 8/19; A61Q 1/12; A61Q 1/02

(52) U.S. Cl. CPC A61K 8/731; A61K 8/19; A61Q 1/12; A61Q 1/02; A61K 2800/5426; A61K 2800/43

(57) ABSTRACT

Provided is a powder cosmetic composition including a positive charge donor material for enhancing makeup skin adhesion and preventing staining. The powder cosmetic composition may provide an effect of excellent makeup skin adhesion by including a positive charge donor material of a specific combination, thereby exhibiting a significantly higher positive charge than when a single positive charge donor material is included.

### TECHNICAL FIELD

The present disclosure relates to a cosmetic composition including a powder containing a positive charge donor material for enhancing makeup skin adhesion and preventing staining.

### BACKGROUND ART

Masks, which were worn intermittently to protect the respiratory tract from air pollutants such as fine dust and yellow dust, have recently become increasingly more important and commonplace due to the spread of SARS, MERS, and novel coronaviruses. As the mask wearing time and frequency are gradually increasing, many problems are occurring, such as makeup crumbling or smearing on the mask due to contact with the mask.

As such, coverage and adhesion are essential elements in makeup cosmetics, but in particular, powder formulations have limitations in improving adhesion due to characteristics of the formulations. In addition, in a powder-type compressed formulation, caking or grazing may occur, and when the product is applied, makeup clumping or makeup separation may occur. As described above, various methods have been tried in order to improve the adhesion and coverage of cosmetics, but the effectiveness is still insufficient, and an effect evident enough to be perceived by consumers has not been exhibited.

In this regard, according to previous studies, the skin surface is known to have a negative charge (see J. Pharm. Sci, 76(10) (1987) 765-773; and J. Controlled Release 103 (2005) 123-136). Therefore, the present inventors confirmed that when a makeup cosmetic composition containing a powder having a positive charge is introduced, strong adhesion to the skin surface may be expected due to electrostatic attraction, and thereby it is possible to prevent smearing on the mask, and thus, the present invention was completed.

### DESCRIPTION OF EMBODIMENTS

### Technical Problem

An aspect provides a cosmetic composition containing a positive charge donor material, wherein in the cosmetic composition, makeup staining is prevented, and skin adhesion is improved.

### Solution to Problem

Provided is a cosmetic composition containing a positive charge donor material, wherein in the cosmetic composition, makeup staining is prevented, and skin adhesion is improved. The cosmetic composition may be a powder cosmetic composition.

The term “positive charge donor substance”, used herein, refers to a substance that provides a positive net charge at a selected pH, such as a physiological pH, wherein the physiological pH may refer to pH 6 to pH 8, specifically, pH 6.5 to pH 8, or more specifically, pH 7.5.

A zeta potential of the positive charge donor material and the powder including the same may exhibit a positive potential, and may exhibit a positive charge of a certain size that allows the powder to adhere to the skin of a negative charge. For example, the zeta potential of the positive charge donor material or the powder including the same may be 5 mV to 120 mV, for example, 5 mV to 110 mV, 5 mV to 100 mV, 5 mV to 95 mV, 5 mV to 90 mV, 10 mV to 120 mV, 10 mV to 110 mV, 10 mV to 100 mV, 10 mV to 95 mV, 10 mV to 90 mV, 20 mV to 120 mV, 20 mV to 110 mV, 20 mV to 100 mV, 20 mV to 95 mV, 20 mV to 90 mV, 30 mV to 120 mV, 30 mV to 110 mV, 30 mV to 100 mV, 30 mV to 95 mV, 30 mV to 90 mV, 40 mV to 120 mV, 40 mV to 110 mV, 40 mV to 100 mV, 40 mV to 95 mV, 40 mV to 90 mV, 45 mV to 120 mV, 45 mV to 110 mV, 45 mV to 100 mV, 45 mV to 95 mV, 45 mV to 90 mV, 45 mV to 80 mV, or 45 mV to 70 mV, under neutral pH conditions. In an embodiment, a zeta potential of a powder not including a positive charge donor material is measured as a negative value, whereas a mixed powder including iron oxide, polyquaternium or a combination thereof is confirmed to have a zeta potential measured as a positive value.

As the positive charge donor material, any kind of a material capable of providing a positive charge may be used, but the positive charge donor material may be, for example, iron-oxide, polyquaternium (for example, polyquaternium-10, polyquaternium-7, etc.), guar hydroxypropyltrimonium chloride, poly-L-lysine, or a combination thereof. The iron oxide may be red iron oxide, yellow iron oxide, black iron oxide, or a combination thereof, but is not limited thereto.

In an embodiment, the positive charge donor material may be a combination of iron oxide and polyquaternium-10. Accordingly, the powder may be a mixed powder containing iron oxide and polyquaternium-10. In an embodiment, when the combination of iron oxide and polyquaternium-10 is used as the positive charge donor material, the combination positive charge donor material exhibits a higher positive charge than a single positive charge donor material, and thus is confirmed to have an excellent skin adhesion improvement effect and makeup staining prevention effect. In an example, it was confirmed that the mixed powder including the combination of iron oxide and polyquaternium-10 exhibits a higher zeta potential, compared to the powder including only iron oxide or polyquaternium-10. In addition, it was confirmed that the mixed powder including the combination of iron oxide and polyquaternium-10 exhibits a higher zeta potential, compared to the mixed powder including a combination of iron oxide and polyquaternium-6.

The powder may include the positive charge donor material in an appropriate amount to obtain an effect of enhanced skin adhesion and prevention of makeup staining.

The powder may include 0.05 wt % to 95 wt %, for example, 0.05 wt % to 90 wt %, for example, 0.5 wt % to 95 wt %, for example 0.5 wt % to 90 wt %, for example, 1 wt % to 95 wt %, for example, 1 wt % to 90 wt %, for example, 5 wt % to 95 wt %, for example, 5 wt % to 90 wt %, for example, 10 wt % to 95 wt %, for example, 10 wt % to 90 wt %, for example, 5 wt % to 85 wt %, for example, 10 wt % to 85 wt %, for example, 15 wt % to 95 wt %, for example, 15 wt % to 90 wt %, for example, 20 wt % to 95 wt % of iron oxide, with respect to the total weight of the powder. When a content of iron oxide increases outside the above range, since iron oxide affects the color of the cosmetic composition, there may be an issue that the desired coloration of the cosmetic composition is difficult, and when a content of iron oxide decreases outside the above range, the effect of enhanced skin adhesion and prevention of makeup staining may be insignificant.

The powder may include 1 wt % to 60 wt %, for example, 1 wt % to 55 wt %, for example, 1 wt % to 50 wt %, for example, 5 wt % to 60 wt %, for example, 5 wt % to 55 wt %, for example, 5 wt % to 50 wt %, for example, 10 wt % to 60 wt %, for example, 10 wt % to 55 wt %, for example, 10 wt % to 50 wt % of polyquaternium-10, with respect to the total weight of the powder. When a content of polyquaternium-10 increases outside the above range, viscosity of the cosmetic composition increases, which may negatively affect the formulation and sensation of application of the cosmetic composition, and when a content of polyquaternium-10 decreases outside the above range, the effect of enhanced skin adhesion and prevention of makeup staining may be insignificant.

The powder may further include a combination of powders included in general powder cosmetics, and may include any powders included in a powder cosmetic composition in the art. For example, the powder may include at least one selected from the group consisting of talc, mica, synthetic mica, sericite, silica, titanium dioxide, silicon-based powder, boron powder, zinc oxide, nylon powder, polymethacrylate powder, urethane powder, acrylate (co)polymer, polyethylene (co)polymer, organic pigments, inorganic pigments and pearlescent pigments. In addition, the powder may not be surface-treated, or may be surface-treated with silicon, metal salt, fatty acid, amino acid, lauroyl lysine or lecithin.

Since the cosmetic composition includes a powder containing a positive charge donor material, skin adhesion of the cosmetic composition is remarkably improved, thereby solving the issue that makeup applied to the skin is smeared on another surface, for example, a mask. In an embodiment, iron oxide and polyquaternium-10 are contained in the powder as positive charge donor materials, and a cosmetic composition including the same is prepared to evaluate a degree of makeup staining, and in the cosmetic composition including iron oxide and polyquaternium-10, the degree of makeup staining is confirmed to be remarkably improved, compared to a cosmetic composition not including the powder.

The cosmetic composition may include the powder in an appropriate amount to obtain an effect of enhanced skin adhesion and prevention of makeup staining. The cosmetic composition may include 0.0001 wt % to 20 wt %, 0.0001 wt % to 10 wt %, 0.0001 wt % to 5 wt %, 0.001 wt % to 20 wt %, 0.001 wt % to 10 wt %, 0.001 wt % to 5 wt %, 0.01 wt % to 20 wt %, 0.01 wt % to 10 wt %, 0.01 wt % to 5 wt %, 0.1 wt % to 20 wt %, 0.1 wt % to 10 wt %, 0.1 wt % to 5 wt %, 1 wt % to 20 wt %, 1 wt % to 10 wt %, or 1 wt % to 5 wt % of the powder, with respect wt % to the wt % total weight of the composition. When a content of the powder is out of the above range, a desired cosmetic formulation may not be obtained, or the effect of improving skin adhesion and preventing makeup staining may be insignificant.

The formulation of the cosmetic composition is not limited as long as it is a formulation of cosmetics in the art, and may be a formulation made by molding cosmetics on a certain plate, for example, a compressed powder, a compressed pact, powder foundation or a powder pact, etc. An oil binder may be included to improve moldability of the powder, and the oil binder may include ester-based oil, hydrocarbon-based oil, and silicon oil.

The cosmetic composition may further include preservatives, stabilizers, surfactants, solubilizers, moisturizers, emollients, UV absorbers, preservatives, bactericides, antioxidants, pH adjusters, organic and inorganic pigments, fragrances, cooling agents or limiting agents, etc. The blending amount of the additional ingredients such as a moisturizing agent may be easily selected by those skilled in the art within the range that does not impair the purpose and effect of the present disclosure.

### Advantageous Effects of Disclosure

A powder cosmetic composition according to an aspect may include a positive charge donor material, thereby solving the issue of makeup staining and providing an effect of enhanced skin adhesion.

A powder cosmetic composition according to another aspect may provide an effect of excellent makeup skin adhesion by including a positive charge donor material of a specific combination, thereby exhibiting a significantly higher positive charge than when a single positive charge donor material is included.

### BRIEF DESCRIPTION OF DRAWINGS

FIG. 1 is a graph showing a surface potential (mV) measurement value of a powder according to Comparative Example 1.

FIG. 2 is a graph showing a surface potential (mV) measurement value of a powder according to Comparative Example 2.

FIG. 3 is a graph showing a surface potential (mV) measurement value of a powder according to Comparative Example 3.

FIG. 4 is a graph showing a surface potential (mV) measurement value of a mixed powder according to Example 1.

FIG. 5 is a graph showing a surface potential (mV) measurement value of a mixed powder according to Example 2.

FIG. 6 is a graph showing a surface potential (mV) measurement value of a powder according to Comparative Example 4.

FIG. 7 is a graph showing a surface potential (mV) measurement value of a powder according to Example 3.

FIG. 8 is an image confirming an effect of enhanced skin adhesion and prevention of mask staining of a cosmetic composition containing a cationic powder.

### BEST MODE

Hereinafter, the present disclosure will be described in more detail through examples. However, these examples are intended to illustrate the present disclosure, and the scope of the present disclosure is not limited to these examples.

### Comparative Examples 1 to 3, and Examples 1 and 2. Preparation of Mixed Powders Containing Positive Charge Donors

In order to prepare powders having a positive charge, powders were mixed and prepared in the compositions of comparative examples and examples shown in Table 1 below. Specifically, Comparative Examples 1 to 3 were prepared to contain iron oxide, silica, or polyquaternium-10, respectively, and Examples 1 and 2 were prepared to contain iron oxide, silica, and polyquaternium-10.

TABLE 1

| | Content (wt %) | | | | |
|---|---|---|---|---|---|
| | Comparative | Comparative | Comparative | | |
| Ingredient | Example 1 | Example 2 | Example 3 | Example 1 | Example 2 |
| Iron oxide | — | 100 | — | 90 | 10 |
| Silica | 100 | — | — | 1 | 50 |
| Polyquaternium-10 | — | — | 100 | 9 | 40 |

### Comparative Example 4 and Example 3. Preparation of Mixed Powders Containing Positive Charge Donors

In order to prepare powders having a positive charge, powders were mixed and prepared in the compositions of comparative examples and examples shown in Table 2 below. Specifically, Comparative Example 4 was prepared to contain black iron oxide and polyquaternium-6, and Example 1 was prepared to contain yellow iron oxide and polyquaternium-10.

TABLE 2

Content (wt %)

| | Comparative | |
|---|---|---|
| Ingredient | Example 4 | Example 3 |
| Black iron oxide | 90 | — |
| Yellow iron oxide | — | 90 |
| Silica | 1 | 1 |
| Polyquaternium-10 | — | 9 |
| Polyquaternium-6 | 9 | — |

### Experimental Example 1. Confirmation of Surface Potential (Zeta Potential)

1.1 Confirmation of Particle Size and Zeta Potential

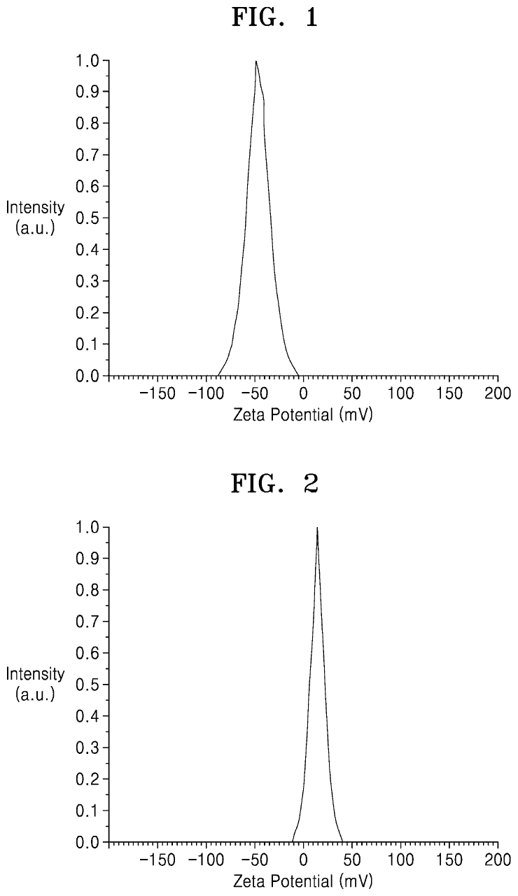

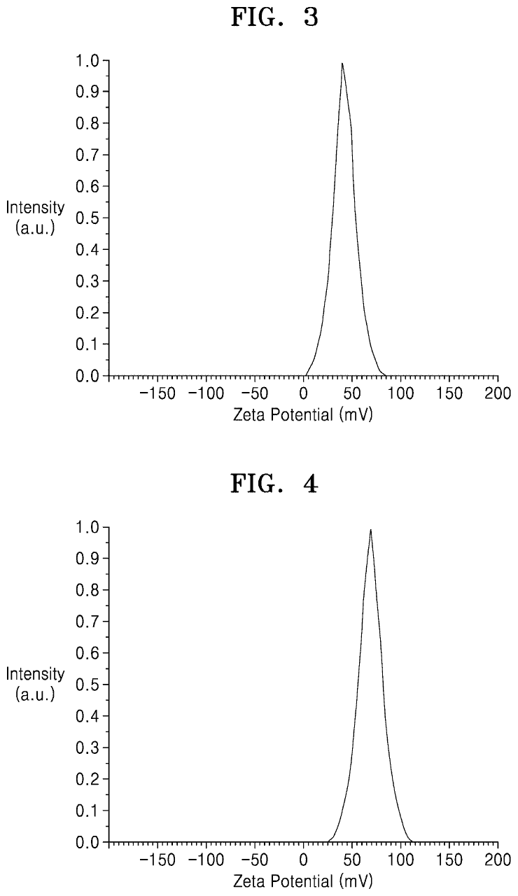

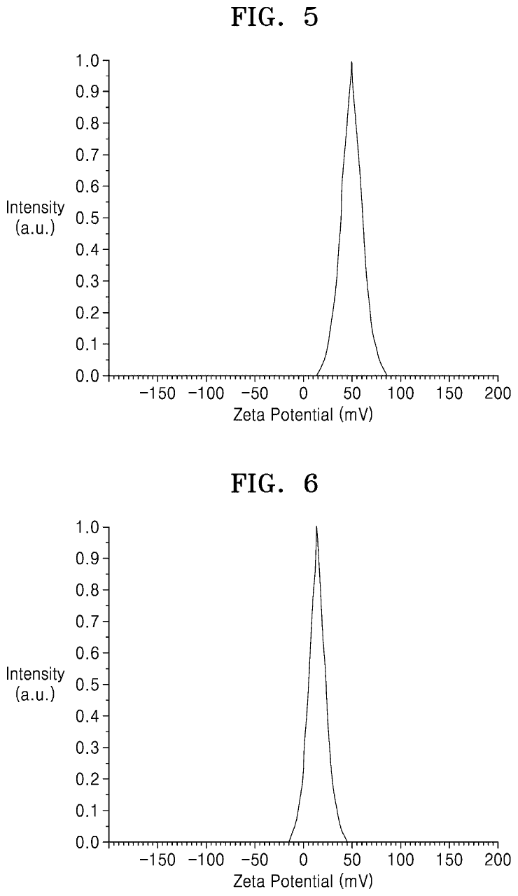

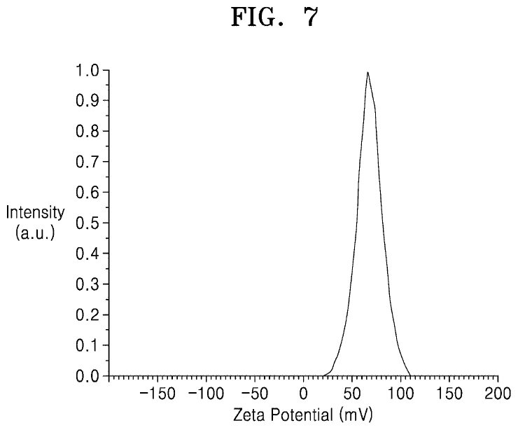

Surface potential of a mixed powder containing a positive charge donor material was measured as follows. Surface potential of the powder was measured by using a dynamic light scattering device (HORIBA, SZ-100) capable of measuring particle sizes and surface potential. Prior to measuring the surface potential of the mixed powder, surface potential of distilled water was first measured, and it was confirmed that the surface potential was zero. As a method of measuring the surface potential of the mixed powder, the mixed powder was uniformly dispersed in water, and then the surface potential was measured by using an SZ-100 analyzer. The measured zeta potential values of the powders of Comparative Examples 1 to 3 and Examples 1 and 2 are shown in FIGS. 1 to 5 and Table 2 below. In addition, the measured zeta potential values of the powders of Comparative Example 4 and Example 3 are shown in FIGS. 6 to 7 and Table 4 below.

TABLE 3

| Experiment | Comparative | Comparative | Comparative | | |
|---|---|---|---|---|---|
| item | Example 1 | Example 2 | Example 3 | Example 1 | Example 2 |
| Zeta potential (mV) | −46.8 | 13.9 | 41.4 | 67.9 | 49.3 |
| | −47.5 | 18.8 | 40.8 | 65.8 | 50.2 |
| | −51.7 | 16.1 | 39.9 | 68.8 | 49.0 |
| | −54.0 | 13.8 | 41.1 | 67.5 | 48.2 |
| | −51.8 | 12.9 | 40.7 | 67.6 | 51.6 |
| Average | −50.4 | 15.1 | 40.8 | 67.5 | 49.7 |

TABLE 4

| Experiment | Comparative | |
|---|---|---|
| item | Example 4 | Example 3 |
| Zeta | 14.8 | 66.1 |
| potential (mV) | 17.7 | 67.5 |
| | 22.5 | 67.7 |
| Average | 18.3 | 67.1 |

As a result, as shown in FIGS. 1 to 5 and Table 3, the powder of Comparative Example 1, which does not contain a positive charge donor material, exhibited a negative charge, whereas the powders of Comparative Examples 2 and 3 containing only iron oxide or polyquaternium-10 had an average surface potential of 15.1 mV and 40.8 mV, respectively, showing a moderate positive charge.

In particular, the surface potentials of the powders of Examples 1 and 2 including a mixture of iron oxide and polyquaternium-10, which are positive charge donor materials, were on average 67.5 mV and 49.7 mV, respectively, showing a significantly increased positive charge compared to the powders of Comparative Examples 2 and 3 including a single positive charge donor material, as well as the powder of Comparative Example 1 not including a positive charge donor material.

In addition, as shown in FIGS. 6 to 7 and Table 4, the mixed powder of Example 3 including a combination of iron oxide and polyquaternium-10 exhibited a remarkably stronger positive charge, compared to the mixed powder of Comparative Example 4 including a combination of iron oxide and polyquaternium-6.

### Comparative Example 5 and Example 4. Preparation of Foundation Containing Positive Charge Donor Substance

In order to investigate the effect of the presence or absence of a mixed power which contains a positive charge donor material on the prevention of staining of a makeup cosmetic composition, foundation cosmetic compositions of Comparative Example 5 and Example 4 were prepared by varying whether or not the mixed powder of Example 1, which has the highest surface potential, was contained.

Specifically, each raw material was weighed in a beaker according to the components and contents shown in Table 5 below, and then uniformly mixed for 5 minutes at 1,000 rpm using a disper at a temperature of 75° C. to 80° C. After mixing, a powder was added at the same temperature and uniformly mixed for 3 minutes at 1,000 rpm. Then, air bubbles were removed and the mixture was cooled to prepare each cosmetic composition. Table 5 below shows specific components and contents of the cosmetic compositions of Comparative Examples 5 and Example 4.

TABLE 5

| | Content (wt %) | |
|---|---|---|
| | Comparetive | |
| Ingredient | Example 5 | Example 4 |
| Lauryl PEG-10 tris(trimethylsiloxy)silylethyl | 3.0 | 3.0 |
| dimethicone | | |
| Phenyl trimethicone | 10.0 | 10.0 |
| Cyclopentasiloxane | 21.0 | 21.0 |
| Ethylhexyl methoxycinnamate | 7.0 | 7.0 |
| Ethylhexyl salicylate | 5.0 | 5.0 |
| Disteadimonium hectorite | 0.5 | 0.5 |
| Particulate titanium dioxide | 7.0 | 7.0 |
| Titanium dioxide | 9.0 | 9.0 |
| Yellow iron oxide | 1.0 | 1.0 |
| Purified water | To 100 | To 100 |
| Disodium EDTA | 0.02 | 0.02 |
| Magnesium sulfate | 0.7 | 0.7 |
| Glycerin | 5.0 | 5.0 |
| Phenoxyethanol | 0.7 | 0.7 |
| Mixed powder containing positive | — | 3.0 |
| charge donor material (Example 1) | | |

### Experimental Example 2. Evaluation of Makeup Staining

For the foundation cosmetic compositions of Example 4 and Comparative Example 5, the degree of makeup staining when wearing a mask was evaluated.

Specifically, after washing, and waiting for 10 minutes under constant temperature and humidity conditions, the cosmetic compositions of Example 4 and Comparative Example 5 were applied to the right and left sides of the face, respectively, and the product was fixed for 5 minutes. Thereafter, the mask was worn, and after 2 hours of daily life, an image of the worn mask that was in contact with the face was taken.

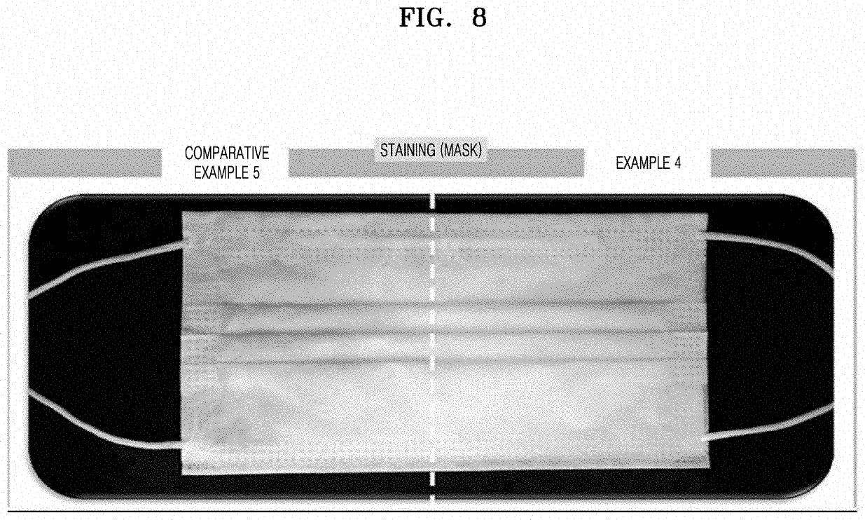

As a result, as shown in FIG. 8, the composition of Example 4 was confirmed to show significantly less mask staining when applied to the skin, compared to the composition of Comparative Example 5. From the above results, it was confirmed that the composition of Example 4, compared to the composition of Comparative Example 5, may increase skin adhesion of the cosmetic composition, by further including a mixed powder containing a positive charge donor material, thereby preventing the problem of staining on the mask.

### Experimental Example 3. Skin Safety Evaluation

Skin safety of the foundation cosmetic compositions of Example 4 and Comparative Example 5 was evaluated.

Specifically, irritation degrees of the cosmetic compositions of Example 4 and Comparative Example 5 were evaluated with 20 men and women without a skin disease as subjects. After applying 20 μL of the sample to the entire arm of the test subjects, the test site was sealed and patched for 24 hours. 30 minutes and 24 hours after removing the patch, the reaction of the skin was examined according to the terminology presented in the Cosmetic, Toiletry and Fragrance Association (CTFA) guidelines. Skin irritation index (PII) scores of the test subjects obtained according to the criteria were averaged, and the irritation was evaluated as mild, when the average was less than 1, moderate, when the average was less than 2, and severe, when the average was 3.5 or more. Skin irritation indexes of the cosmetic compositions of Example 4 and Comparative Example 5 are shown in Table 6 below.

TABLE 6

| Experiment | Comparative | |
|---|---|---|
| item | Example 5 | Example 4 |
| Skin irritation index (PII) | No irritation | No irritation |

As a result, as shown in Table 6, it was confirmed that both compositions of Comparative Example 5 and Example 4 may be safely used as cosmetic compositions without irritation. From the above results, it was confirmed that even when the mixed powder containing the positive charge donor material was further included, skin safety was not adversely affected.

### Experimental Example 4. Sensory Evaluation

Sensory evaluation (5-point scale) was performed on the foundation cosmetic compositions of Example 4 and Comparative Example 5.

Specifically, sensation of application, feeling of adhesion, and overall satisfaction were evaluated as very good 5 points, good 5 points, moderate 3 points, bad 2 points, and very bad 1 point. The sensory evaluation results of the cosmetic compositions of Example 4 and Comparative Example 5 are shown in Table 7 below.

TABLE 7

| | Comparative | |
|---|---|---|
| Item | Example 5 | Example 4 |
| Sensation of application | 4.0 | 4.0 |
| Feeling of adhesion | 3.0 | 4.5 |
| Overall satisfaction | 3.7 | 4.4 |

As a result, as shown in Table 7, it was confirmed that the cosmetic composition of Example 4 maintained an excellent degree of sensation of application similar to the composition of Comparative Example 5, and was significantly improved in terms of feeling of adhesion and overall satisfaction, compared to the composition of Comparative Example 5. From the above results, it was confirmed that the composition of Example 4 exhibited remarkably improved feeling of adhesion of the makeup and overall satisfaction, compared to the composition of Comparative Example 5, without negatively affecting the sensation of application, by including a mixed powder containing a positive charge donor material. From the above results, it was confirmed that the cosmetic composition including the positive charge donor material significantly increased skin adhesion and could solve the problem of makeup staining, by including a positive charge donor material, in particular, by including a composition of iron oxide and polyquaternium-10.

1. A cosmetic composition comprising a powder comprising a positive charge donor material, wherein the cosmetic composition prevents makeup staining and has improved skin adhesion.

2. The cosmetic composition of claim 1, wherein the positive charge donor material is iron oxide, polyquaternium-10, polyquaternium-7, guar hydroxypropyltrimonium chloride, poly-L-lysine, or a combination thereof.

3. The cosmetic composition of claim 1, wherein the powder is a mixed powder comprising iron oxide and polyquaternium-10.

4. The cosmetic composition of claim 3, wherein the powder comprises 0.05 wt % to 95 wt % of iron oxide.

5. The cosmetic composition of claim 3, wherein the powder comprises 1 wt % to 50 wt % of polyquaternium-10.

6. The cosmetic composition of claim 1, wherein the powder exhibits a zeta potential of 40 mV to 120 mV.

7. The cosmetic composition of claim 1, comprising 0.0001 wt % to 20 wt % of the powder, with respect to the total weight of the composition.
